(19) United States
(12) Patent Application Publication
(10) Pub. No.: US 2006/0122162 A1
(43) Pub. Date: Jun. 8, 2006

(54) Methods of using temozolomide formulation intrathecally in the treatment of cancers

(75) Inventor: David L. Cutler, Moorestown, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/290,779

(22) Filed: Nov. 30, 2005

Related U.S. Application Data
(60) Provisional application No. 60/632,675, filed on Dec. 2, 2004.

(51) Int. Cl. A61K 31/33

(52) U.S. Cl. 514/183

(57) ABSTRACT

Methods are disclosed for treating cancer in a patient in need of such treating comprising intrathecally administering temozolomide in a pharmaceutical formulation in a therapeutically effective amount.

This application claims priority from U.S. Provisional Application No. 60/632,675, filed Dec. 2, 2004, the entirety of which is incorporated by reference as if set forth fully herein.

### FIELD OF THE INVENTION

The present invention relates to methods of using pharmaceutical formulations comprising temozolomide intrathecally in the treatment of cancers. This application specifically discloses intrathecal methods of using pharmaceutical compositions comprising temozolomide or a pharmaceutically acceptable salt thereof, at least one aqueous diluent, and at least one dissolution enhancing agent sufficient to substantially dissolve temozblomide or the pharmaceutically acceptable salt thereof, wherein said dissolution enhancing agent is urea, L-histidine, L-threonine, L-asparagine, L-serine, L-glutamine, or a combination of two or more of the above.

### BACKGROUND OF THE INVENTION

Antineoplastic agents are useful in cancer therapies against a wide array of cancer and other diseases. Temozolomide (described in U.S. Pat. No. 5,260,291) is one such antineoplastic agent.

Temozolomide is known for its anti-tumor effects. For example, one study showed that clinical responses were achieved in 17% of patients having advanced melanoma (Newlands et al., Br J Cancer, 65(2):287-291 (1992)). In another study, a clinical response was achieved in 21% of patients with advanced melanoma (Bleehan et al., Journal of Clinical Oncology, 13(4):910-913 (1995)). Treatment of gliomas in adults with temozolomide is also known (O'Reilly et al., Eur J Cancer, 29A(7):940-942 (1993) and Eur J Cancer, 29A(10):1500 (1993)). Treatment of the following cancers in adults with temozolomide has also been disclosed: metastatic melanoma; malignant melanoma, high grade glioma, glioblastoma and other brain cancers; lung cancer; breast cancer; testicular cancer; colon and rectal cancers; carcinomas; sarcomas; lymphomas; leukemias; anaplastic astrocytoma; and mycosis fungoides.

Temozolomide is a water-insoluble compound. Temozolomide has been administered intrathecally in patients as micronized suspensions, as disclosed in U.S. Pat. No. 6,251,886. However, suspension formulations may not always be desirable in such administrations.

WO03/072082 published Sep. 4, 2003, discloses pharmaceutical formulations comprising temozolomide or a pharmaceutically acceptable salt thereof, at least one aqueous diluent, and at least one dissolution enhancing agent sufficient to substantially dissolve temozolomide or the pharmaceutically acceptable salt thereof, wherein said dissolution enhancing agent is urea, L-histidine, L-threonine, L-asparagine, L-serine, L-glutamine, or a combination of two or more of the above, as being useful for intravenous methods of treating cancer. In addition to intravenous methods, it would also be highly desirable to provide intrathecal methods of treating cancer.

### SUMMARY OF THE INVENTION

It has now been found that pharmaceutical formulations comprising temozolomide or a pharmaceutically acceptable salt thereof, at least one aqueous diluent, and at least one dissolution enhancing agent sufficient to substantially dissolve temozolomide or the pharmaceutically acceptable salt thereof, wherein said dissolution enhancing agent is urea, L-histidine, L-threonine, L-asparagine, L-serine, L-glutamine, or a combination of two or more of the above, can be useful for intrathecal methods of treating cancer. In particular, the present invention provides a method for treating cancer in a patient in need of such treatment comprising administering temozolomide or a pharmaceutically acceptable salt thereof intrathecally in a therapeutically effective amount, wherein the temozolomide is administered as a pharmaceutical formulation comprising, in addition to temozolomide, at least one aqueous diluent, and at least one dissolution enhancing agent sufficient to substantially dissolve temozolomide or the pharmaceutically acceptable salt thereof, wherein said dissolution enhancing agent is urea, L-histidine, L-threonine, L-asparagine, L-serine, L-glutamine, or a combination of two or more of the above.

In a preferred embodiment, the above-described pharmaceutical formulations comprising temozolomide can be administered intrathecally to the subarachnoid space, and the intrathecal administration can be intralumbar or intraventricular (via, for example, the Ommaya reservoir).

Thus, the present invention offers a unique method of administering a temozolomide-containing pharmaceutical formulation intrathecally to a patient to treat cancer.

The present invention also provides methods of intrathecally administering temozolomide formulation in combination with an ATase inhibitor. Preferably, the Atase inhibitor is O⁶-benzylguanine, 8-oxo-O⁶-benzylguanine, 4-bromothenylguanine, or a pharmaceutically acceptable salt of any of these agents. In one embodiment, the ATase inhibitor is administered orally. In another embodiment, the ATase inhibitor is administered intrathecally. In yet another embodiment, the ATase inhibitor is administered intravenously.

The present invention also provides methods of intrathecally administering temozolomide formulation in combination with a nitrosourea oncolytic agent. Preferably, the nitrosourea oncolytic agent is GLIADEL® Wafer.

The present invention also provides methods of administering temozolomide formulation orally or intravenously in combination with an ATase inhibitor wherein the ATase inhibitor is administered intrathecally. Preferably, the Atase inhibitor is O⁶-benzylguanine, 8-oxo-O⁶-benzylguanine, 4-bromothenylguanine, or a pharmaceutically acceptable salt of any of these agents.

The present invention also provides methods of administering dacarbazine orally or intravenously in combination with an ATase inhibitor wherein the ATase inhibitor is administered intrathecally. Preferably, the Atase inhibitor is O⁶-benzylguanine, 8-oxo-O⁶-benzylguanine, 4-bromothenylguanine, or a pharmaceutically acceptable salt of any of these agents.

### DESCRIPTION OF THE INVENTION

U.S. Pat. No. 6,251,886 describes methods of using microcrystalline formulations of temozolomide to treat various cancers, by administering to a patient in need thereof. The administration can be by methods described therein, including intrathecal administration.

WO03/072082 published Sep. 4, 2003, discloses pharmaceutical formulations comprising temozolomide or a pharmaceutically acceptable salt thereof, at least one aqueous diluent, and at least one dissolution enhancing agent sufficient to substantially dissolve temozolomide or the pharmaceutically acceptable salt thereof, wherein said dissolution enhancing agent is urea, L-histidine, L-threonine, L-asparagine, L-serine, L-glutamine, or a combination of two or more of the above, as being useful for intravenous methods of treating cancer.

The term “temozolomide” is intended to mean a compound having the formula I: 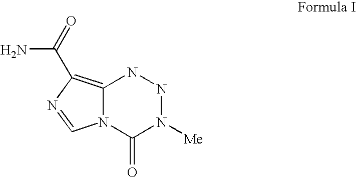
One chemical name for temozolomide is 3,4-dihydro-3-methyl-4-oxoimidazo-[5,1-d]-1,2,3,4-tetrazin-8-carboximide. Another chemical name is 8-carbamoyl-3-methylimidazo[5,1-d]-1,2,3,5-tatrazin-4(3H)-one). The synthesis of temozolomide is well known. See, for example, Stevens et al., J Med Chem, 27(2):196-201 (1984) and Wang et al., J Chem Soc, Chem Commun, 1994, pp 1687-1688. Temozolomide is available commercially from Schering-Plough Corporation, Kenilworth, N.J. (e.g., under the trade name TEMODAR® in the U.S. and TEMODAL® in Europe).

The temozolomide-comprising compositions useful in the present intrathecal administration are the same that are described in the above-cited WO03/072082, published Sep. 4, 2003, which is incorporated herein by reference. WO03/072082 discloses lyophilized pharmaceutical formulations (as well as diluted or reconstituted formulations) comprising temozolomide or a pharmaceutically acceptable salt thereof, at least one aqueous diluent, and at least one dissolution enhancing agent sufficient to substantially dissolve said temozolomide or the pharmaceutically acceptable salt thereof, wherein said dissolution enhancing agent is urea, L-histidine, L-threonine, L-asparagine, L-serine, L-glutamine, or a combination of two or more of the above, as being useful for intravenous methods of treating cancer. A preferred dissolution enhancing agent is L-histidine. The term “temozolomide formulation” in the present application refers to the formulation disclosed in WO03/072082.

When urea is used in the pharmaceutical formulation as the dissolution enhancing agent, its weight percent (wt %) in the pharmaceutical formulation can range from about 4 wt % to about 60 wt %, preferably from about 8 wt % to about 30 wt %, more preferably from about 12 wt % to about 22 wt %. When L-histidine, L-threonine, L-asparagine, L-serine, L-glutamine, or a combination of two or more of the above are used in the pharmaceutical formulation as the dissolution enhancing agent(s), its wt % in the pharmaceutical formulation can range from about 2 wt % to about 60 wt %, preferably from about 4 wt % to about 40 wt %, more preferably from about 8 wt % to about 20 wt %.

In addition to temozolomide and the dissolution-enhancing agent, the pharmaceutical formulation can further comprise at least one excipient. Non-limiting examples of suitable excipients include polysorbates, polyethylene glycols (PEG), propylene glycols, polysorbates, or a combination of two or more of the above. When an excipient is used in the pharmaceutical formulation, its wt % in the pharmaceutical formulation can range from about 1 wt % to about 50 wt %, preferably from about 2 wt % to about 30 wt %, more preferably from about 4 wt % to about 16 wt %. A preferred excipient is a polysorbate or PEG.

The pharmaceutical formulation may further comprise at least one bulking agent. Non-limiting examples of suitable bulking agents which can be included in the pharmaceutical formulation include mannitol, lactose, sucrose, sodium chloride, trehalose, dextrose, starch, hydroxyethylstarch (hetastarch), cellulose, cyclodextrins, glycine, or a combination of two or more of the above. In a preferred embodiment, the bulking agent in the pharmaceutical formulation-is mannitol. When a bulking agent is used in the pharmaceutical formulation, its wt % in the pharmaceutical formulation can range from about 20 wt % to about 80 wt %, preferably from about 35 wt % to about 65 wt %, more preferably from about 40 wt % to about 56 wt %.

In another embodiment, the pharmaceutical formulation may further comprise at least one buffer. Non-limiting examples of suitable buffers which can be included in the pharmaceutical formulation include citrate buffers, lithium lactate, sodium lactate, potassium lactate, calcium lactate, lithium phosphate, sodium phosphate, potassium phosphate, calcium phosphate, lithium maleate, sodium maleate, potassium maleate, calcium maleate, lithium tartarate, sodium tartarate, potassium tartarate, calcium tartarate, lithium succinate, sodium succinate, potassium succinate, calcium succinate, lithium acetate, sodium acetate, potassium acetate, calcium acetate, or a combination of two or more of the above. Preferably, a buffer used in the pharmaceutical formulation is at least one citrate buffer such as, for example, lithium citrate monohydrate, sodium citrate monohydrate, potassium citrate monohydrate, calcium citrate monohydrate, lithium citrate dihydrate, sodium citrate dihydrate, and the like. When a buffer is used in the pharmaceutical formulation, its wt % in the pharmaceutical formulation can range from about 5 wt % to about 60 wt %, preferably from about 10 wt % to about 40 wt %, more preferably from about 15 wt % to about 28 wt %.

In another embodiment, the pharmaceutical formulation may further comprise a pH adjuster. Non-limiting examples of suitable pH adjusters which can be included in the pharmaceutical formulation are hydrochloric acid, sodium hydroxide, citric acid, phosphoric acid, lactic acid, tartaric acid, succinic acid, or a combination of two or more of the above. A preferred pH adjuster for the pharmaceutical formulation is hydrochloric acid. When a pH adjuster is used in the pharmaceutical formulation, its wt % in the pharmaceutical formulation can range from about 1 wt % to about 20 wt %, preferably from about 2 wt % to about 12 wt %, more preferably from about 4 wt % to about 8 wt %.

In another embodiment, the pharmaceutical formulation useful in the present invention comprises (i) temozolomide in an amount ranging from about 1 wt % to about 50 wt %, (ii) hydrochloric acid in an amount ranging from about 1 wt % to about 20 wt %, (iii) a citrate buffer in an amount ranging from about 5 wt % to about 60 wt %, (iv) a polysorbate in an amount ranging from about 1 wt % to about 50 wt %, (v) L-histidine, L-threonine, L-asparagine, L-serine, L-glutamine, or a combination of two or more of the above, in an amount ranging from about 2 wt % to about 60 wt %, and (vi) mannitol in an amount ranging from about 15 wt % to about 85 wt %.

The pH of the pharmaceutical formulation useful for intrathecal administration preferably ranges from about 2.5 to about 6.0, more preferably from about 3.0 to about 4.5, and most preferably from about 3.8 to about 4.2.

The lyophilized pharmaceutical formulation can additionally be reconstituted (resolubilized) in a volume of at least one aqueous diluent, if so desired. The lyophilized formulations of the pharmaceutical formulations can be diluted or reconstituted prior to administration with a suitable aqueous diluent(s) to obtain a finished concentration.

The term “aqueous diluent(s)” means aqueous fluids suitable for injection into a patient. Non-limiting examples of aqueous diluents include water, normal saline, 5% dextrose solution, and other fluids suitable for intrathecal administration into a patient.

The term “pharmaceutically acceptable salts” refers to suitable acid addition salts as well as salts providing the anion of the quaternary salt include those prepared from acids such as hydrochloric, hydrobromic, phosphoric, sulfuric, maleic, citric, acetic, tartaric, succinic, oxalic, malic, glutamic, pamoic and the like, and other acids related to the pharmaceutically acceptable salts listed in J Pharm Sci, 66:2 (1977).

The term “effective amount” or “therapeutically effective amount” shall mean that amount of active ingredient that will elicit the biological, clinical or medical response of a tissue, system, or animal that is being sought by a researcher or clinician.

The term “patient” is intended to mean animals, mammals, humans, monkeys, rodents, domestic and farm animals. The term “therapeutically effective amount” is intended to mean an amount of a therapeutic agent of the composition, such as temozolomide or other antineoplastic agents described above, that will have an antineoplastic effect on a tissue, system, animal or mammal that is being sought by the administrator (such as a researcher, doctor, or veterinarian), which includes alleviation of the symptoms of the condition or disease being treated and the prevention, slowing or halting of progression of the neoplastic condition.

The term “weight percent” (“wt %”) for purposes of this invention is calculated on a basis of total weight of the pharmaceutical formulation.

Another aspect of the invention relates to a process for treating or preventing disease in a patient comprising administering a therapeutically effective amount of the above-described pharmaceutical formulation to a patient in need thereof, by way of intrathecal administration. Non-limiting examples of diseases which can be treated or prevented include carcinoma, sarcoma, melanoma, glioma, glioblastoma, brain cancer, lung cancer, thyroid follicular cancer, pancreatic cancer, breast cancer, anaplastic astrocytoma, bladder cancer, myelodysplasia, prostate cancer, testicular cancer, colon and rectal cancer, lymphoma, leukemia, or mycosis fungoides.

The dosage regimen utilizing the pharmaceutical formulations of the invention is selected based upon consideration of a variety of factors, including species, age, weight, sex and medical condition of the patient; the specific disease to be treated, the severity of the condition to be treated; the route of administration; the renal and hepatic function of the patient; and the particular active ingredient or salt thereof employed. An ordinarily skilled physician can readily determine and prescribe the effective amount of antineoplastic agent required to prevent, counter, or arrest the progress of the disease condition.

Specific dosing regimens may vary depending on the route of administration. For example, in a non-limiting way of describing, while preferred doses of temozolomide administered from the instantly described formulation by intrathecal administration are from about 0.1 to about 400 mg, doses are more preferably from about 1 to about 100 per single intrathecal administration, still more preferably about 5 to about 25 mg per single intrathecal administration. Preferably, a final temozolomide concentration of 100 μM to 25 mM, more preferably 250 μM to 5 mM, is achieved in the cerebrospinal fluid (CSF). A preferred dosing schedule for intrathecal administration is once a day, but can include less frequent dosing. For example, on day 1, a dose of 5 to 10 mg can be administered as an initial dose, and 24 hours later another dose can be administered. Then, for example, treatments can be administered twice a week for two weeks in a row, then once a week for two consecutive weeks, followed by once a week every other week for two treatments, then one treatment per month thereafter. Alternately, dosing less frequent than once a day may also be utilized. Thus, for example, intralumbar administration or one intraventrical administration may be given once a week for 4 weeks followed by once every 2-4 weeks.

Also encompassed within the scope of the present invention are methods of administering temozolomide intrathecally according to the methods taught herein in combination with an ATase inhibitor (e.g., O⁶-benzylguanine (O⁶BG), 8-oxo-O⁶-benzylguanine (8-oxo-O⁶BG), 4-bromothenylguanine (4-BTG, PaTrin-2, Patrin™, Lomeguatrib; KuDOS Pharmaceuticals Ltd., Cambridge, England), or a pharmaceutically acceptable salt of any of these agents.

In a preferred embodiment, the ATase inhibitor is administered in therapeutically effective amount (e.g., about 1-2000 mg/kg ATase inhibitor, and preferably about 10-800 mg/kg). The ATase inhibitor may be administered p.o., I.V., or intrathecally. Notably, Berg et al. (Berg et al., Clin Cancer Res, 4(11):2891-2894 (1998)) describe the pharmacokinetics of O⁶BG and its active metabolite 8-oxo-O⁶BG in cerebrospinal fluid (CSF) and plasma after intrathecal administration of O⁶BG in a nonhuman primate model.

The preferred dosage of 4-BTG is 10 mg/m², administered p.o., I.V., or intrathecally. An exemplary dosage regimen for 4-BTG is described in Middleton et al., Proceedings of the 11^{th} NCI•EORTC•AACR Symposium, No. 538 (2000)). In short, the dosage regimen exemplified is as follows: 10 mg/m² 4-BTG daily for five days, followed by a two week break, and subsequent administration of 10 mg/m² 4-BTG daily for five days every 4 weeks.

Also encompassed within the scope of the present invention are methods of administering temozolomide according to the methods taught herein in combination with a nitrosourea oncolytic agent. In a preferred embodiment, the nitrosourea oncolytic agent is GLIADEL® Wafer (polifeprosan 20 with carmustine implant; Guilford Pharmaceuticals Inc., Baltimore, Md., USA). The preferred dosage of GLIADEL® Wafer is eight wafers, wherein each wafer contains 7.7 mg of carmustine, resulting in a dose of 61.6 mg when eight wafers are implanted.

In another embodiment, TMZ may be administered p.o. or I.V. (e.g., as described in U.S. Pat. No. 5,731,304) in combination with intrathecal administration of one or more of the following: an ATase inhibitor (e.g., O⁶BG, 8-oxo-O⁶BG, 4-BTG), or a nitrosourea oncolytic agent (e.g., GLIADEL® Wafer).

In yet another embodiment, DTIC-Dome® ((dacarbazine), Bayer Pharmaceuticals Corp., West Haven, Conn., USA) may be administered I.V. in combination with intrathecal administration of one or more of the following: an ATase inhibitor (e.g., O⁶BG, 8-oxo-O⁶BG, 4-BTG), or a nitrosourea oncolytic agent (e.g., GLIADEL® Wafer). Recommended dosages for DTIC in the treatment of malignant melanoma and Hodgkin's disease are described below. For malignant melanoma, the recommended dosage of DTIC-Dome is 2 to 4.5 mg/kg/day for 10 days; wherein treatment may be repeated at 4 week intervals. An alternative recommended dosage for malignant melanoma is 250 mg/square meter body surface/day I.V. for 5 days; wherein treatment may be repeated every 3 weeks. For Hodgkin's disease, the recommended dosage of DTIC-Dome is 150 mg/square meter body surface/day for 5 days, in combination with other effective drugs; wherein treatment may be repeated every 4 weeks. An alternative recommended dosage for Hodgkin's disease is 375 mg/square meter body surface on day 1, in combination with other effective drugs, to be repeated every 15 days.

Examples of Human Treatment Regimens

Set forth below are specific treatment regimens for intrathecal temozolomide formulation administered via intralumbar or intraventricular routes. These specific treatment regimens are for illustrative purposes only and should not be construed as limiting the scope of the present invention in any way. In these regimens, each dose is repeated on a twice a week basis (i.e., q 3-4 days) for a total of 4 doses. In general, the same route of administration is used throughout each specific treatment regimen. The term “drug” in the following description of administration refers to the temozolomide in the pharmaceutical formulation described in WO03/072082.

Administration:

Intralumbar Administration: LP

For patients receiving the drug via the intralumbar route, the drug should preferably be administered in a final total volume of 10 ml in an appropriate diluent for injection, USP. Drug administration is preferably isovolumetric (i.e., an amount of CSF equivalent to that to be administered must be removed prior to drug injection). Following intralumbar injection, patients should lie supine either in the flat or Trendelenburg position for approximately 30 minutes.

Intraventricular Administration: Ommaya Reservoir:

After appropriate sterile preparation of the reservoir site, the drug should preferably be administered into the Ommaya reservoir via a 23 gauge (or smaller) scalp vein needle in a final total volume of 10 ml in an appropriate diluent for injection, USP. Drug administration is preferably isovolumetric (i.e., an amount of CSF equivalent to that to be administered must be removed from the reservoir prior to drug injection). The drug should be injected slowly at a rate not exceeding 2 ml/min. Following administration of the drug, the reservoir should be flushed slowly for 1 to 2 minutes with approximately 2 cc of either CSF (removed prior to drug injection) or 0.9% saline. After the flush injection, the reservoir should be pumped 4 to 6 times.

Unless there is surgical or clinical contraindication, patients will be treated via intraventricular administration (Ommaya reservoir). Starting Dose: For adults (age >18 years), the starting dose of the drug under this particular regimen is preferably from about 1 mg to about 400 mg/dose, more preferably about 5 mg to about 100 mg by single intrathecal injection. The dose could be higher or lower, depending upon the patient's condition and the need as determined by a physician. The administration could be by direct intrathecal injection, or slow infusion using an appropriate apparatus (such as, for example, an Ommaya reservoir or an infusion pump).

Criteria for Subsequent Treatment:

If ANC is≧1500/mm³ and platelet count is ≧100,000/mm³ (absent use of growth factors to enhance levels), then repeat doses may be administered otherwise additional temozolomide is delayed. If subsequent administrations cannot be administered on the scheduled day of dosing, the CBC can be repeated weekly for up to and including 3 weeks until the ANC is >1500/mm³ and platelet count >100,000/mm³. If these hematological criteria are met, the drug may be administered.

Modifications and variations of this invention will be apparent to those skilled in the art. The specific embodiments described herein are offered by way of example only, and the invention is not to be construed as limited thereby.

What is claimed is:

1. A method of treating a cancer sensitive to cytotoxic effects of temozolomide in a patient in need of such treatment, comprising intrathecally administering to the patient an effective amount of a pharmaceutical formulation comprising temozolomide or a pharmaceutically acceptable salt thereof, at least one aqueous diluent, and at least one dissolution enhancing agent sufficient to substantially dissolve temozolomide or the pharmaceutically acceptable salt thereof, wherein said dissolution enhancing agent is urea, L-histidine, L-threonine, L-asparagine, L-serine, L-glutamine, or a combination of two or more of the above.

2. The method according to claim 1, wherein said pharmaceutical formulation further comprises an excipient selected from the group consisting of polysorbate, polyethylene glycol, propylene glycol, polypropylene glycol, or a combination of two or more of the above.

3. The method according to claim 1, wherein said pharmaceutical formulation further comprises at least one bulking agent selected from the group consisting of mannitol, lactose, sucrose, sodium chloride, trehalose, dextrose, starch, hetastarch, cellulose, cyclodextrins, glycine, or a combination of two or more of the above.

4. The method according to claim 1, wherein said pharmaceutical formulation further comprises at least one buffer selected from the group consisting of lithium citrate monohydrate, sodium citrate monohydrate, potassium citrate monohydrate, calcium citrate monohydrate, lithium citrate dihydrate, sodium citrate dihydrate, potassium citrate dihydrate, calcium citrate dihydrate, lithium citrate trihydrate, sodium citrate trihydrate, potassium citrate trihydrate, calcium citrate trihydrate, lithium citrate tetrahydrate, sodium citrate tetrahydrate, potassium citrate tetrahydrate, calcium citrate tetrahydrate, lithium citrate pentahydrate, sodium citrate pentahydrate, potassium citrate pentahydrate, calcium citrate pentahydrate, lithium citrate hexahydrate, sodium citrate hexahydrate, potassium citrate hexahydrate, calcium citrate hexahydrate, lithium citrate heptahydrate, sodium citrate heptahydrate, potassium citrate heptahydrate, calcium citrate heptahydrate, lithium lactate, sodium lactate, potassium lactate, calcium lactate, lithium phosphate, sodium phosphate, potassium phosphate, calcium phosphate, lithium maleate, sodium maleate, potassium maleate, calcium maleate, lithium tartarate, sodium tartarate, potassium tartarate, calcium tartarate, lithium succinate, sodium succinate, potassium succinate, calcium succinate, lithium acetate, sodium acetate, potassium acetate, calcium acetate, or a combination of two or more of the above.

5. The method according to claim 1, wherein said pharmaceutical formulation further comprises a pH adjuster selected from the group consisting of hydrochloric acid, sodium hydroxide, citric acid, phosphoric acid, lactic acid, tartaric acid, succinic acid, or a combination of two or more of the above.

6. The method according to claim 1, wherein the pH of said pharmaceutical formulation ranges from about 2.5 to about 6.0.

7. The method of claim 1, wherein the pharmaceutical formulation is intrathecally administered to a subarachnoid space or cavity inside of the patient.

8. The method of claim 7, wherein from about 0.1 to about 400 mg of temozolomide is administered to the patient by a single intrathecal injection.

9. The method of claim 8, wherein from about 1 to about 100 mg of temozolomide is administered to the patient by a single intrathecal injection.

10. The method of claim 9, wherein from about 5 to about 25 mg of temozolomide is administered to the patient by a single intrathecal injection.

11. The method of claim 7, wherein said administration is intralumbar.

12. The method of claim 7, wherein said administration is intraventricular.

13. The method of claim 1, wherein the cancer is carcinoma, sarcoma, melanoma, glioma, glioblastoma, brain cancer, lung cancer, thyroid follicular cancer, pancreatic cancer, breast cancer, anaplastic astrocytoma, bladder cancer, myelodysplasia, prostate cancer, testicular cancer, colon and rectal cancer, lymphoma, leukemia, or mycosis fungoides.

14. The method of claim 13, wherein the cancer is a primary brain tumor or a lymphoma.

15. The method of claim 14, wherein the primary brain tumor is a medulloblastoma, glioma, or glioblastoma.
